SEQUENCE LISTING (18 sequences)

SEQ ID NO: 1 - DNA, 261 bp, Homo sapiens
tttgtgaaccaacacctgtgcggctcacacctggtggaagctctctacctactgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcctggcattgtggaacaatgctgtaccagcatctgctccctctaccagctggagaactactgcaactag

SEQ ID NO: 2 - DNA, 261 bp, Yeast
ttcgttaaccaacacttgtgtggttctcacttggttgaagctttgtacttggtttgtggtgaaagaggtttcttctacactccaaagactagaagagaagctgaagacttgcaagttggtcaagttcaattgggtggtggtccaggtcctggttctttgcaaccattggctttggaaggttctttggaaaagagaggtattgttgaacaatgttgtacttctatttgttctttgtaccaattggaaaactactgtaactaa

SEQ ID NO: 3 - DNA, 100 bp, Artificial
  synthesized oligonucleotides
gcattacgtattcgttaaccaacacttgtgtggttctcacttggttgaagctttgtacttggtttgtggtgaaagaggtttcttctacactccaaagact

SEQ ID NO: 4 - DNA, 104 bp, Artificial
  synthesized oligonucleotide
atatgcggccgcttagttacagtagttttccaattggtacaaagaacaaatagaagtacaacattgttcaacaatacctcttttagtctttggagtgtagaaga

SEQ ID NO: 5 - DNA, 184 bp, Artificial
  PCR product
gcattacgtattcgttaaccaacacttgtgtggttctcacttggttgaagctttgtacttggtttgtggtgaaagaggtttcttctacactccaaagactaaaagaggtattgttgaacaatgttgtacttctatttgttctttgtaccaattggaaaactactgtaactaagcggccgcatat

SEQ ID NO: 6 - DNA, 36 bp, Artificial
  synthesized oligonucleotides
atctctcgagaaaagattcgttaaccaacacttgtg

SEQ ID NO: 7 - DNA, 36 bp, Artificial
  synthesized oligonucleotides
atctgaattcatcttaagtctttggagtgtagaaga

SEQ ID NO: 8 - DNA, 122 bp, Artificial
  PCR product
atctctcgagaaaagattcgttaaccaacacttgtgtggttctcacttggttgaagctttgtacttggtttgtggtgaaagaggtttcttctacactccaaagacttaagatgaattcagat

SEQ ID NO: 9 - DNA, 36 bp, Artificial
  synthesized oligonucleotides
atctctcgagaaaagaggtattgttgaacaatgttg

SEQ ID NO: 10 - DNA, 36 bp, Artificial
  synthesized oligonucleotides
atctgaattcatctagttacagttagttttccaatt

SEQ ID NO: 11 - DNA, 94 bp, Artificial
  PCR product
atctctcgagaaaagaggtattgttgaacaatgttgtacttctatttgttctttgtaccaattggaaaactactgtaactagatgaattcagat

SEQ ID NO: 12 - DNA, 30 bp, Artificial
  synthesized oligonucleotides
atctgacgtcagatctaacatccaaagacc

SEQ ID NO: 13 - DNA, 30 bp, Artificial
  synthesized oligonucleotides
atctgacgtcaagcttgcacaaacgaactt

SEQ ID NO: 14 - DNA, 71 bp, Artificial
  synthesized oligonucleotides
gctactcgagaaaagattcgttaaccaacacttgtgtggttctcacttggttgaagctttgtacttggttt

SEQ ID NO: 15 - DNA, 70 bp, Artificial
  synthesized oligonucleotides
tagcgcggccgcttaagtctttggagtgtagaagaaacctctttcaccacaaaccaagtacaaagcttca

SEQ ID NO: 16 - DNA, 90 bp, Artificial
  PCR product
ttcgttaaccaacacttgtgtggttctcacttggttgaagctttgtacttggtttgtggtgaaagaggtttcttctacactccaaagact

SEQ ID NO: 17 - DNA, 90 bp, Artificial
  synthesized oligonucleotides
gctactcgagaaaagaggtattgttgaacaatgttgtacttctatttgttctttgtaccaattggaaaactactgtaagcggccgcgcta

SEQ ID NO: 18 - DNA, 90 bp, Artificial
  synthesized oligonucleotides
tagcgcggccgcttacagtagttttccaattggtacaaagaacaaatagaagtacaacattgttcaacaatacctcttttctcgagtagc

(19) United States
(12) Patent Application Publication
(10) Pub. No.: US 2009/0017496 A1
(43) Pub. Date: Jan. 15, 2009

(54) Co-expression of multiple protein chains or subunits

(76) Inventors: Yangao Ma, Hubei (CN); Xiangdong Ma, North Andover, MA (US)

Correspondence Address: Duan Wu, 165 Friend St. #5, Boston, MA, 02114, US

(21) Appl. No.: 11/667,158

(22) PCT Filed: Oct. 11, 2005
(86) PCT No.: PCT/US05/36479

§ 371 Date: May 3, 2007

(30) Foreign Application Priority Data
Nov. 3, 2004 (CN) 200410061039.0

(51) Int. Cl. C12P 21/04; C12N 15/11; C12N 15/00; C07K 14/00

(52) U.S. Cl. 435/69.4; 536/23.51; 530/303; 435/320.1

(57) ABSTRACT

A recombinant genetic construct is provided that includes at least two expression cassettes. Each cassette encodes for a chain or subunit of a target protein. The genetic construct preferably targets any expressed protein to the secretory pathway of the host cell. An application of present invention is found in expressing the two chains of human insulin through two separate expression cassettes on the same methylotrophic yeast expression vector. Mature, bioactive human insulin molecules are secreted through this method without resorting to any post-translational cleavage process.

### CROSS-REFERENCE TO RELATED APPLICATIONS

This application claims priority to and the benefit of Chinese patent application Serial No. 200410061039.0, filed Nov. 3, 2004, the entire contents of which are incorporated by reference herein.

### TECHNICAL FIELD

The invention generally relates to a process and related constructs and systems for producing proteins through recombinant DNA techniques. More particularly, the invention employs a host cell to co-express at least two separate chains, subunits or their equivalents of a target protein, e.g., human insulin, through a single recombinant expression vector. Preferably, the host cell is selected to secret the protein in a bioactive form. The invention has advantageous applications in, for instance, large-scale pharmaceutical manufacturing.

### BACKGROUND OF THE INVENTION

To optimize a recombinant technique-based biological process for protein production, e.g., for pharmaceutical use, cost efficiency is a critical consideration. To that end, the fewer steps and the fewer preparations of reagents used during the process the better. Existing biotech-based manufacturing processes often include one or more of the following problems. First, many steps of modification may be required of an intermediary before a satisfactory end product can be obtained. Second, the living organism used in the process often produces the desired intermediaries and/or end product only during a certain and very limited period of its life cycle, and therefore, compromising the yield. Third, the desired intermediaries and/or end product often require costly and multi-stage purification.

Currently, one way to manufacture proteins that have multiple, separate chains or subunits is to produce various chains or subunits first, and then purify, modify and assemble them ex vivo into the final form. While these chains or subunits are not linked by the same polypeptide chain in the final product, they often need to be assembled according to an exact ratio and under appropriate conditions to form the natural, bioactive configuration. Such conditions may include the correct oxidation/reduction states of specific amino residues in the chains or subunits in order to form the needed cross-linking, e.g., disulfide bonds, that stabilizes the protein. Under such a scenario, incorrect ratio between the chains or subunits often results in insoluble or inactive products that seriously compromise the yield and quality of the process.

Alternatively, multiple chains and subunits may be manufactured as one polypeptide with or without a linker polypeptide. This approach remedies the incorrect ratio problem mentioned above to an extent, but a cleavage step is required to separate the chains and subunits before they can be properly assembled into the final product. The cleavage step, chemically or enzymatically based, plus post-cleavage modification steps are again time-consuming and costly.

For example, the stable and bioactive form of insulin consists of two polypeptide chains commonly referred to as the A chain and the B chain. Human insulin, a representative example of the insulin family has an A chain of 21 amino acids and a B chain of 30 amino acids. Both chains are covalently connected by two disulphide bridges between A and B chains and a third disulphide bond located within the A chain. See e.g., G. Bell, et al. Nature 282: 525-527 (1979). The two chains of insulin, in their natural production, are encoded by a single mRNA, translated into a polypeptide, and then cleaved into separate chains. In that sense, the A, B chains of insulin are not subunits as in some other macromolecules because they are naturally produced as one piece and only separate from each other through a cleavage step after translation. As will be described further below, manufacturers have used both aforementioned approaches to make insulin.

An example of a polypeptide with subunits instead of the kind of chains found in insulin is interleukin-12 (IL-12). IL-12 consists of two polypeptide subunits (p35 and p40), but the two subunits represent two distinct and unrelated gene products—from 3p12-3q13.2(p35) and 5q31-q33(p40), respectively—linked by disulphide bonds (Gubler U. et al. Co-Expression Of Two Distinct Genes Is Required To Generate Secreted Bioactive Catatonic Lymphocyte Maturation Factor. Proc. Natl. Acad. Sec. USA, 88: 4143-47 (1991)).

For pharmaceutically important polypeptides that have chains or subunits, attempts at their large-scale production have always faced the problems described above. Therefore, there exists a need for a simpler and more efficient manufacturing process that significantly reduces steps needed for modification, purification or cleavage, and yet at the same time, supplies high-yield and high-quality product despite the ratio requirement on component chains and subunits. The industry of insulin manufacture is one illustrative example.

Insulin, made specifically by beta cells in the Islets of Langerhans in the pancreas, is known to be the only natural hormone that reduces blood sugar level, and hence, a remedy for diabetes. Diabetes mellitus is a serious and often debilitating disease that affects over 18.2 million, or 6.3% of the United States population. In China, 30 million people suffer from diabetes. Characterized by an under-utilization of glucose and an absolute or relative insulin deficiency, persons suffering from the disease have a tendency to develop hyperglycemia, glycosuria, and ultimately atherosclerosis, neuropathy, nephropathy and microangiopathy. Diabetes is the seventh leading cause of death in the United States. It is also the leading cause of new cases of blindness in persons of 20-74 years of age and the leading cause of end-stage renal disease. Moreover, diabetic patients are at much greater risk for amputation, heart disease and stroke than the general population.

There are two types of diabetes: Type I and Type II. Type I diabetes results if the beta cells degenerate so the body cannot make enough insulin on its own. A person with this type of diabetes must receive an external source of insulin in order to survive. In Type II diabetes, the beta cells produce insulin, but cells throughout the body do not respond normally to it. Insulin medication also may be used in Type II diabetes to help overcome cells' resistance to insulin. Among adults with diagnosed diabetes, about 30% require daily insulin dosage either through injections, pumps, or other means of intake. Diabetes is a chronic disease that a cure is yet to come to light.

Porcine and bovine insulin were initially used to treat diabetes. However, long-term use of animal insulin poses significant drawbacks. After prolonged use, patient's body may generate immune response against animal insulin, resulting not only in reduced efficacy but also inflammatory responses at injection sites. In addition, production of animal insulin is not projected to be able to keep up with the steadily increasing new cases of diabetes worldwide.

In 1982, genetically engineered human insulin was approved for use to treat diabetes, bringing many additional benefits. Unlike animal insulin, genetically engineered human insulin causes no side effect and it is much easier to control its quality. Further, there are abundant supplies of raw materials. Recently, a variety of insulin analogs have been devised by changing certain amino acids in the wild type insulin. Some of these insulin analogs have clinical significance because they possess the bioactivity found in wild type insulin, yet are less likely to aggregate and, therefore, potentially more advantageous in clinical applications.

In natural production, the human insulin gene first expresses a preproinsulin that can be represented by: prepropeptide-B-C-A. The prepropeptide has 24 amino acids and functions as an exporting signal sequence. The C peptide is a connector peptide of 31 amino acids between the B and A chains. Attracted by the receptor located on the endoplasmic reticulum, a newly produced prepropeptide penetrates the membrane and goes into the cavity of the endoplasmic reticulum. There, the signal sequence is cut off by trypsin and carboxypeptidase B at two base amino acids. Then, the remainder of the preproinsulin, called proinsulin, goes all the way through the Golgi body where the C peptide is split and the A and B chains get properly folded. The A and B chains then finally emerge as a mature insulin molecule. The biochemical process of natural insulin production is well documented in the art and can be found in references such as Steiner et al. Clin. Invest. Med. 9:328-36. (1986).

Presently, there are mainly two approaches in insulin production in the pharmaceutical industry. According to the first method, the A and B chains of the human insulin gene are separately expressed in host microorganisms, particularly, Escherichia coli (E. coli). After purification, the two polypeptides expressed by the host are assembled in vitro into an insulin molecule by chemically forming the disulphide bridges between the two chains A and B through an oxidizing process. This method has several drawbacks—most importantly is the formation of random disulfide bridges on the two chains, generating molecules with incorrect tertiary structures. The proclivity for forming random disulphide bridges is so great that the yield of native insulin with biological activity is driven down and the production costs are driven up dramatically. Further, using E. coli as a host limits the potential for yield—due to its small size, E. coli simply cannot hold a relatively large gene such as the human insulin gene very well.

In the second method, the proinsulin gene is cloned and expressed in a host microorganism, resulting in a single polypeptide that includes chains B and A, linked by the C peptide (B-C-A). This method is premised on the observation that the C peptide apparently plays a role, in nature, in making the cysteines on the A chain and B chain spatially favored for forming the correct disulfide bridges (Bell et al., Nature 284: 26-32 (1980)). After expression and secretion, the C peptide is cleaved in vitro, resulting in separate A and B chains. Although various methods have been devised to shorten the length of the C peptide, a post-translational cleavage step is always required of this method, which is time-consuming and costly. Further details of above-mentioned methods can be found in references such as U.S. Patent Application 20030104607 by N. Annibali, incorporated herein by reference. In short, there remains a need for a simpler and more economical method for the production of recombinant drugs such as insulin and insulin analogs.

### SUMMARY OF THE INVENTION

The present invention relates to methods and related constructs and systems where a single recombinant genetic construct includes at least two expression cassettes. Each cassette encodes for a chain or subunit of a target protein. Each cassette has its own 5′ regulatory region, whether they are of the same or different sequences.

The recombinant genetic construct of the present invention produces at least two chains or subunits when the construct is expressed in a host cell. Preferably, each expression cassette in the genetic construct includes a leader sequence that transports its respective, translated polypeptide through a desired processing pathway, such that the chains or subunits of the target protein that are part of the translated polypeptides are processed in vivo before being secreted from the host cell.

In one embodiment, the host cell is selected such that the cell is capable of performing multiple post-translational modifications desired of the target protein. The polypeptide preferably is secreted from the host cell in a bioactive form. This may require folding the polypeptide into the correct tertiary structure having, for example, disulphide bridges at correct positions. This may also require the host cell to proteolitically process and/or glycosilate the target protein before secreting it into the surrounding medium.

In one embodiment, the present invention provides a simple and efficient way to produce a target protein that has at least two chains that are formed through post-translational cleavage in natural production. By constructing a vector with a number of expression cassettes that correspond to the correct number of each chain or subunit in the target protein, the present invention ensures that separate chains, in the correct ratio, are being sent through the cellular processing machinery to make the desired target protein. As a result, there is no more need for any linking peptide between the chains just to spatially favor formation of the correct structure. Without any linking peptide, there is no more need for post-translational cleavage to separate the chains or the related purification, modification steps. In short, the present invention can dramatically simplify the production and improve the yield, for example, in the production of insulin and its analogs.

Application of the present invention is not limited to production of proteins that, in nature, mature after some of its chains are cleaved post-translationally. The present invention can also be used to produce proteins with multiple subunits that are naturally expressed separately. For example, the present invention can be used to produce cytokines such as IL-12 using a vector that includes separate expression cassettes that encode separate subunits of IL-12.

Therefore, in one aspect, the present invention is directed to a recombinant genetic construct for expressing a target protein that has at least two chains formed by post-translational cleavage in natural production. The recombinant genetic construct includes at least two expression cassettes, each expression cassette including a sequence substantially corresponding to a chain in the target protein. The translation of the recombinant genetic construct expresses the target protein without the post-translational cleavage required in natural production. In one embodiment, the target protein is mammalian in nature. In one feature, the genetic construct of the present invention consists of DNA or RNA.

In another aspect, the present invention is directed to the protein expressed by the above recombinant genetic construct.

In yet another aspect, the present invention is directed to a cell that has been transformed with the above recombinant genetic construct. The cell is capable of expressing the target protein in a bioactive form, e.g., its natural folding configuration, without the post-translational cleavage otherwise required in natural production. In one embodiment, at least one disulfide bond has been formed between the recombinantly expressed chains to form the target protein upon secretion. In another embodiment, the target protein has been glycosilated upon secretion.

In a further aspect, the present invention is directed to a method for producing a bioactive target protein. The method includes providing the above cell transformed with the above recombinant genetic construct, and expressing, through the cell, the target protein in a bioactive form without the post-translational cleavage required in natural production.

In yet another further aspect, the present invention is directed to a recombinant DNA comprising the sequence of:

Pm₁-Ld₁-Pt₁-Y₁-Tm₁-Pm₂-Ld₂-Pt₂-Y₂-Tm₂,

where each listed element is operably linked to an adjacent element, Pm stands for a yeast promoter sequence, Ld stands for a yeast leader sequence, Pt stands for a protease recognition sequence, and Tm stands for a yeast termination sequence. In one embodiment, Y₁ and Y₂ stand for DNA sequences for B chain and A chain of human insulin, respectively. In another embodiment, Y₁ and Y₂ stand for, respectively, DNA sequences for two subunits of a protein, e.g., a cytokine such as IL-12.

In one aspect, the present invention is directed to a recombinant human insulin molecule produced by: providing a eukaryotic cell including a recombinant genetic construct that has a first expression cassette and a second expression cassette, the first expression cassette has a sequence substantially corresponding to the A chain of the human insulin molecule, the second expression cassette has a sequence substantially corresponding to the B chain of the human insulin molecule; inducing the cell to express, through the recombinant genetic construct, the recombinant human insulin molecule and to secret the expressed recombinant human insulin molecule into a surrounding culture; and harvesting the secreted human insulin molecule from the surrounding culture. In one embodiment, the secreted recombinant human insulin molecule is bioactive. An exemplary eukaryotic cell is a yeast cell.

### BRIEF DESCRIPTION OF THE DRAWINGS

The foregoing, and other features and advantages of the invention, as well as the invention itself, will be more fully understood from the description and drawings that follow.

FIG. 1 is a graphic, base-to-base lineup of the DNA sequence of human natural proinsulin B-C-A (SEQ ID NO:1) (top line) against the yeast preferential codon sequence of human proinsulin (SEQ ID NO:2) (bottom line), with base differences underlined. The 5′ and 3′ regions, marked in bold, are coding sequences for B and A chains of the proinsulin, respectively. The middle region, in regular font, is the coding sequence for the C-peptide.

FIG. 2 is a diagram depicting how two oligonucleotides “5′-USAp” (top) and “3′-USAp” (bottom) used in a step in Example 1 anneal to each other due to base complementarity. Expected extension in both directions during PCR is depicted in dotted lines.

FIG. 3 is photographic representation of an agarose gel demonstrating the size of a PCR product obtained after Step 1 in Example 1. The PCR marker lane shows standard bands at 1K, 0.75K, 0.5K, 0.3K, 0.15K and 50 bps, respectively. Lanes 1 and 2 were each loaded with the expected B-C′-A sequences of human proinsulin analog as synthesized by PCR. Lanes 3 and 5 were each loaded with oligonucleotide “5′-USAp”; and lanes 4 and 6 were each loaded with oligonucleotide “3′-USAp”.

FIG. 4 is a diagram illustrating the structure of a vector based on a yeast shuttle vector pPIC9K and expressing the human pro-insulin analog B-C′-A. The depicted expression vector pPIC9K(B-C′-A) was constructed as an intermediary for further vector building.

FIG. 5 is a diagram depicting a strategy used in Example 1 to construct vector pPIC9K(+B+A) from intermediary plasmid pPIC9K(B-C′-A).

FIG. 6 is a restriction map of intermediary plasmid pPIC9K(+B) expressing B chain of human insulin (marked as “B”).

FIG. 7 is a restriction map of intermediary plasmid pPIC9K(+A) expressing A chain of human insulin (marked as “A”).

FIG. 8 is a restriction map of the final expression plasmid pPIC9K(+B+A) expressing both B and A chains of human insulin.

FIG. 9 is a photographic representation of an Immunodotting Blot analysis. IN1, IN3, IN5 in the far left column are human insulin standards in increasing concentrations. The remaining columns each contain samples from culture media of transformed host cells; the samples were collected at different time points, specifically, at 0, 24, 48, 72, 96, and 120 hours, respectively. Each row contained samples from the same yeast colony with the bottom row “9K” containing samples from the culture media of cells transformed by the original plasmid pPIC9K.

FIG. 10 is a photographic representation of a Western Blot analysis. The far right “IN” lane shows a human insulin standard. Lanes 1, 2, 3, 4 each depict assay results of surrounding media samples collected at 24, 48, 72 and 96 hours of fermentation, respectively, from transformed colonies.

FIG. 11 includes three diagrams of HPLC analyses. The top diagram labeled as “2” depicts the elution profile of a sample collected from culture media of transformed host cells at 96 hours of fermentation. The middle diagram labeled as “2+IN” depicts the elution profile of the sample from “2” with the addition of human insulin standard sample. The bottom diagram labeled “IN” depicts the elution profile of a human insulin standard sample.

### DETAILED DESCRIPTION OF THE INVENTION

Unless otherwise indicated, all terms used herein have the same meaning as they would to one skilled in the art of the present invention. Practitioners are particularly directed to Sambrook et al. (1989) Molecular Cloning: A Laboratory Manual (Second Edition), Cold Spring Harbor Press, Plainview, N.Y. and Ausubel F M et al. (1989) Current Protocols in Molecular Biology, John Wiley & Sons, New York, N.Y., for definitions and terms of the art. It is to be understood that this invention is not limited to the particular methodology, protocols, and reagents described, as these may vary.

The term “polypeptide” or “chain” or “subunit,” as used herein, refers to a compound made up of a single succession of amino acid residues linked by peptide bonds. The term “protein” as used herein may be synonymous with the term “polypeptide,” or may refer, in addition, to a complex of two or more polypeptides. As used herein, “subunits” refer to portions of a protein, which portions are or are derived from distinct mRNAs in nature. “Chains,” in contrast, refer to portions of a protein, which portions are or are derived from the same mRNA in nature. Chains are formed, in nature, through post-translational cleavage.

The term “bioactive” or “biologically active” refers to a recombinant or synthetic protein or polypeptide having structural, regulatory or biochemical function of its naturally occurring counterpart.

The term “heterologous” used in the context of nucleotides refers to nucleotides that are not endogenous to the cell or part of the genome in which they are present; generally such nucleotides have been added to the cell, by transfection, microinjection, electroporation, or the like. Such nucleotides generally include at least one coding sequence, but this coding sequence need not be expressed.

The term “genetic construct” as used herein refers to any structure or sequence that effects genetic expression, such as any number of polynucleotides that may be in the form of RNA or in the form of DNA, and include mRNA, cRNA, synthetic RNA and DNA, cDNA, genomic DNA, and PNAs and other antisense RNA and DNA analogs. The genetic construct may be double-stranded or single-stranded, and if single-stranded may be the coding strand or the non-coding (anti-sense, complementary) strand. A genetic construct can be the whole or part of an expression vector.

The term “expression vector” refers to vectors that have the ability to incorporate and express heterologous nucleotides in a host cell.

An “expression cassette,” sometimes referred to as a transcription unit in the art, is used herein to mean a unit in an expression vector in which a nucleotide sequence encoding a protein or protein component, often heterologous, is operably linked to suitable regulatory or control sequences capable of affecting the expression of such protein or protein component in the intended host. Generally, eukaryotic regulatory sequences include a transcriptional promoter, however, it may be appropriate that a sequence encoding suitable mRNA ribosomal binding sites be provided, and, optionally, sequences which control the termination of transcription (termination sequence).

Nucleotide regions are “operably linked” or “operably associated” when they are functionally related to each other. For example: a promoter is operably linked to a coding sequence if it controls the transcription of the sequence; a ribosome binding site is operably linked to a coding sequence if it is positioned so as to permit translation. Generally, operably linked means contiguous and, in the case of leader sequences, contiguous and in reading phase. However, operably linked elements may be spaced apart and have intervening elements.

The term “C-peptide” is used to mean the connection portion of the B-C-A polypeptide sequence of a single-chain proinsulin-like molecule. Specifically, the C-peptide connects position 30 of the B chain and position 1 of the A chain.

The present invention provides an improved cell-based manufacturing process that utilizes an expression system with better yields and greater simplicity, and employs novel gene expression techniques. In one feature, expression vectors are constructed to include multiple expression cassettes that each encodes a distinct component found in a target protein, and a host is selected such that its cellular machinery is manipulated to express these distinct components and process them into a biologically active form, skipping many of the purification, modification and cleavage steps found in present industrial productions.

### Expression Vector

In order to recombinantly produce a target protein that has multiple polypeptide components (polymeric), the present invention seeks to provide, or mimic, simultaneous, and/or proximate translation of such components (chains or subunits) in the correct ratio found in the natural form of the target protein. Previous attempts at using a single promoter to control the co-expression of multiple components of a polymeric protein have faced numerous difficulties. The yield has been disappointing to start with, possibly because of intrinsic limitations on expression efficiency as the size of the heterologous DNA sequence grows under the control of a single promoter. Moreover, because a single promoter or expression cassette expresses the heterologous protein in a single polypeptide chain, complicated steps to cleave the single chain into chains or subunits and to remove any linking peptides are required.

Accordingly, the present invention constructs an expression vector that has at least two expression cassettes, each cassette encoding a distinct chain or subunit found in the target protein. Expression efficiency of each chain or subunit can improve markedly when each is under the control of a separate promoter, whether it is the same kind of promoter or not. The expression cassettes may be spaced from each other on the vector or immediately adjacent (contiguous) to each other. In one embodiment, multiple expression cassettes have the same promoter sequence, equalizing expression efficiency for each chain or subunit encoded by separate expression cassettes.

The resulting vector, by itself, may encode the entire or part of the target protein. For example, if a single molecule of the mature target protein has a copy of each of three distinct components (chains or subunits) A, B and C, an expression vector with two expression cassettes, one encoding component A and the other encoding component B, encodes two thirds of the target protein. In the same example, if the expression vector has three expression cassettes, one encoding component A, another encoding component B, and the third encoding component C, then the expression vector encodes the entire protein. In one embodiment of the invention, the expression cassettes are constructed to correspond to the correct ratio of polypeptide components in the target protein. For example, if there are twice the amount of component A than component B in the mature protein, two expression cassettes each encoding component A and one expression cassette encoding component B may be incorporated into the expression vector of the present invention.

Each expression cassette should include various regulatory elements, starting with a 5′ regulatory region that affects the expression of downstream sequences in the cassette. Generally, eukaryotic regulatory region includes a transcriptional promoter, however, it may be appropriate that a sequence encoding suitable mRNA ribosomal binding sites be provided. Optionally, each expression cassette ends with a sequence that controls the termination of transcription (termination sequence or terminator). In a preferred embodiment of the invention, coding sequence encoding the desired polypeptide component is operably linked to both the 5′ regulatory region and the 3′ termination sequence. Also optionally, an expression cassette may include one or more enhancer sequences to augment the expression of the coding sequence. Because many enhancer sequences are position-independent, they can be positioned elsewhere on the expression vector and outside the expression cassettes, upstream or downstream. All or some of the regulatory elements, such as the entire 5′ regulatory regions, the promoters, the termination sequences, and the optional enhancer sequences in multiple expression cassettes may be the same or different. In a particular embodiment where the 5′ regulatory regions, i.e., the promoters and, optionally, the mRNA ribosomal binding sites, are the same in each expression cassette, transcriptional and translational controls are equalized for the encoded polypeptide components such that uniform co-expression is favored.

To manipulate a host cell into processing any polypeptide expressed by such an expression vector, each expression cassette preferably includes a leader sequence as part of the expression cassette. The leader sequence translates into a signal peptide that facilitates the heterologous polypeptide component to enter a desired intracellular processing pathway, e.g., by translocating the translated polypeptide into the endoplasmic reticulum and then Golgi apparatus, and eventually secreted. In a preferred embodiment, once the signal peptide is recognized and processed by the host cell, the peptide provides the secretion of the target protein into the culture medium. Preferably, the signal peptide is cleaved off in the course of this process or shortly thereafter. To ensure that, the expression cassette may encode a sequence that is recognized by a protease for cleavage. An example of such a protease recognition sequence is amino acid sequence Lysine-Arginine, which is recognized by endoprotease Kex2 for cleavage between Arginine and the next downstream amino acid. The signal peptide-encoding leader sequence, as well as any other element found in the genetic construct of the invention may be heterologous or homologous to the host organism producing the protein.

Once a suitable clone of the target protein's polypeptide component (chain or subunit) has been obtained, whether it is cDNA-based or genomic, inserting its sequence into the expression cassette may be performed by techniques generally known to those of skill in recombinant expression. Preferably, the clone is based on codons preferred by the intended host cell lines. In one embodiment, all the elements in expression cassettes are the same except for the sequence for the heterologous polypeptide component. As a result, expression conditions for various polypeptide components of the target protein are synchronized and harmonized to the maximum to encourage simultaneous and equal-amount co-expression.

According to this aspect of the invention, in an embodiment, a recombinant genetic construct of the invention has multiple expression cassettes that each has the sequence as follows:

Pmₙ-Ldₙ-Ptₙ-Yₙ-Tmₙ

where Pm stands for a promoter sequence, Ld stands for a leader sequence, Pt stands for a protease recognition sequence, Y stands for sequences encoding a distinct chain or subunit of the target protein, Tm stands for a termination sequence and “n” is an indexing integer. Each listed element is operably linked to an adjacent element. Obviously, there may or may not be additional sequences in between elements listed in the formula, and an element may overlap with an adjacent element or include the entire adjacent element. For example, the protease recognition sequence (Pt) may be part of the leader sequence (Ld).

Where the target protein is a dimer having two distinct polypeptide chains or subunits, whether they are the same (homodimer) or different (heterodimer), an embodiment of the expression cassette of the invention may have the following sequence:

Pm₁-Ld₁-Pt₁-Y₁-Tm₁-Pm₂-Ld₂-Pt₂-Y₂-Tm₂,

where Y₁ and Y₂ stand for respective sequences for distinct chains or subunits of the target protein.

Any suitable expression vector can be used to carry the recombinant genetic construct of the present invention. Many prokaryotic and eukaryotic expression vectors are commercially available. Selection of appropriate expression vectors as the starting point of making a genetic construct is within the knowledge of those skilled in the art. Generally speaking, vectors useful for practicing the present invention include plasmids, viruses (including bacteriophage), and integratable DNA fragments (i.e., fragments integratable into the host genome by genetic recombination). In other words, expression cassettes may have one or more copies stably integrated into the particular genome of a host or may be present extra chromosomally on a multicopy vector or on a minichromosomal element.

Most vectors include various regulatory elements. For example, useful yeast vectors may contain an origin of replication from the endogenous 2 micron yeast plasmid or an autonomously replicating sequence (ARS) which confers on the plasmid the ability to replicate at high copy number in the yeast cell, centromeric (CEN) sequences which limit the ability of the plasmid to replicate at only low copy number in the yeast cell, a promoter, DNA encoding the heterologous DNA sequences, sequences for polyadenylation and transcription termination, and a selectable marker gene.

The vector may replicate and function independently of the host genome, as in the case of a plasmid, or may integrate into the genome itself, as in the case of an integratable DNA fragment. In a preferred embodiment, the vector contains replicon and regulatory sequences that are derived from species compatible with the intended expression host. For example, a promoter operable in a host cell is one that binds the RNA polymerase of that cell, and a ribosomal binding site operable in a host cell is one which binds the endogenous ribosomes of that cell.

The expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli. Inserting the constructed expression cassettes into the expression vector is known to one skilled in the art of recombinant expression, and is illustrated below in examples. Appropriate cloning and expression vectors for use with prokaryotic and eukaryotic hosts are also described in Sambrook, et al. (supra).

In a preferred embodiment of the present invention, a yeast vector is used. Any promoter capable of functioning in yeast systems may be selected for use in the constructs and cells. Suitable promoting sequences in yeast vectors include the promoters for metallothionein, 3-phosphoglycerate kinase (PGK) or other glycolytic enzymes such as enolase, glyceraldehyde-3-phosphate dehydrogenase, hexokinase, pyruvate, decarboxylase, phosphofructokinase, glucose-6-phosphate isomerase, 3-phosphoglycerate mutase, pyruvate kinase, triosephosphate isomerase, phosphoglucose isomerase, and glucokinase. Suitable vectors and promoters for use in yeast expression are further described in R. Hitzeman et al., EPO Publication No. 73,657.

Other yeast promoters, which have the additional advantage of transcription controlled by growth conditions, are the promoter regions for alcohol dehydrogenase, 1,2,-isocytochrome C, acid phosphates, degradative enzymes associated with nitrogen metabolism, and the aforementioned metallothionein and glyceraldehyde-3-phosphate dehydrogenase, as well as enzymes responsible for maltose and galactose utilization, such as the galactose inducible promoter, GAL1. Particularly preferred here is the alcohol dehydrogenase promoter AOX1. Finally, in constructing suitable expression plasmids, the termination sequences associated with these genes may also be ligated into the 3′ end of the heterologous coding sequences to provide polyadenylation and termination of the mRNA. In preparing the preferred expression vectors of the present invention, translational initiation sites may be chosen to confer the most efficient expression of a given nucleic acid sequence in the yeast cell (see Cigan, M. and Donahue, T. F., Gene, 59: 1-18 (1987), for a description of suitable translational initiation sites).

### Host Cell

The present invention is preferably practiced on eukaryotic cell lines because prokaryotic cells do not possess the cellular machinery to properly modify after translation, or to fold the expressed polypeptides. Among the numerous known modifications which may be present include, but are not limited to, acetylation, acylation, amidation, ADP-ribosylation, glycosylation, GPI anchor formation, covalent attachment of a lipid or lipid derivative, methylation, myristlyation, pegylation, prenylation, phosphorylation, ubiquitination, or any similar process.

In one embodiment, the expression vector is introduced into eukaryotic cells where chains or subunits encoded by various expression cassettes are expressed in the cells and targeted through the cells' natural secretory pathway. These cells are able to secret mature proteins correctly folded with the correct composition of chains or subunits. In one embodiment, yeast cells are used as host cells.

Yeast has a secretory mechanism that is similar to the secretory system of mammals, including the capacity of folding, of proteolitically processing, of glycosilation and secretion, in a proper manner, the mammalian protein. When appropriate vectors are employed in the yeast for exporting the protein outside the cell, the process of recovering and purification of the proteins exported to the culture medium is simpler and has a better yield relative to the expression in cell cytoplasm. In addition, the secretion system provides an appropriate environment for the formation of the disulfide bridges that are necessary for the folding of the proteins (Smith, et al. Science 229:1219 (1985)). On the other side, the cytoplasm is a reducing environment wherein these connections are not produced. Under these circumstances, the proteins that need disulfide bridges for maintaining a correct tertiary structure, as it is in the case of insulin, can be produced with better results when the same are secreted.

Among the various yeast species, Saccharomyces cerevisiae has been used as hosts to produce a large number of proteins. Usually protein expression utilizes the S. cerevisiae mating factor (α-factor) which consists of a signal sequence (pre) followed by the pro-sequence. The pre sequence consists of 19 amino acids, and the pro sequence consists of 66 amino acids, which include three N-glycoside and one dibasic Kex2 endoprotease processing site that can be recognized by its double basic amino acid residues (Waters et al., JBC, 263: 6209-14 (1988)). However, present methods using S. cerevisiae often face yield problems due to low efficiency promoters and premature proteolysis, among other reasons.

Methylotrophic yeasts, such as Pichia pastoris, offer an unusual advantage in practicing the present invention. These unicellular microorganisms are advantageous hosts for producing heterologous proteins in large volumes. They can grow in the presence of methanol as the only carbon source in the absence of glucose, and can be kept without inconveniences in high density when cultured in high-volume fermentor. It has been shown that expression systems based on methylotrophic yeast can achieve 10-100 times higher yield than other systems depending on the target proteins. For example, Pichia pastoris cells can grow to high-density condition, and have an alcohol dehydrogenase promoter, AOX1, which is strictly controlled by methanol. As a result, expression of heterologous proteins can be induced by methanol without slowing down cell growth. In addition, methylotrophic yeasts are capable of producing many of the pos-translated modifications carried out by the upper eukaryotic cells, such as proteolytic digestions, protein folding, disulfide-bridge formation and glycosilation, making them ideal candidates for expression systems that support industrial production.

Pichia pastoris is one of the twelve species within the four yeast genera capable of metabolizing methanol as the only carbon source (Cregg, J. et al. Bio/Technology 11: 905-910 (1993)). The mechanism of secretion in P. pastoris is similar to S. cerevisiae (Wang, Y. et al., Biotechnology & Bioengineering 73: 74-79 (2001)). Another exemplary methylotrophic yeast expression system is Hansenula polymorpha. The four methylotrophic yeast genera are Pichia, Candida, Hansenula and Torulopsis. It should be understood that besides methylotrophic yeast species, other yeast species such as S. cerevisiae and Kluyveromyces lactis, can also be used to practice the present invention.

Once a cell line has been selected as the expression system or host, one or more copies of the expression vectors carrying the recombinant genetic constructs of the invention are introduced into the host cell. Accordingly, the present invention also relates to host cells which are genetically engineered with vectors of the invention, and the production of proteins and polypeptides of the invention by recombinant techniques. Host cells are genetically engineered (i.e., transduced, transformed or transfected) with the expression vectors of this invention. Preferably, the expression vectors contain one or more selectable marker genes so that host cells that have been genetically engineered successfully can be selected.

The engineered host cells can be cultured in conventional nutrient media modified as appropriate for activating promoters, selecting transformants or amplifying the gene coded by the heterologous sequence. The culture conditions, such as temperature, pH and the like, are those previously used with the host cell selected for expression, and will be apparent to those skilled in the art. Harvesting and verification of the expressed protein or polypeptides including separation, purification, modification, and assembly are also within the conventional knowledge of those skilled in the art.

Features of the present invention are further illustrated by the following non-limiting examples.

### EXAMPLE 1

In order to produce a bioactive human insulin comprising an A chain and a B chain, a recombinant DNA expression vector was constructed to include two expression cassettes to be expressed in a yeast host. The vector was a yeast shuttle vector called pPIC9K, a portion of which was constructed with the following formula:

Pm-Ld-Pt-Y₁-Tm-Pm-Ld-Pt-Y₂-Tm,

where Y₁ is the coding sequence for A chain or B chain while Y₂ is for the other chain of human insulin. Because the intended host was methylotrophic yeast P. pastoris, yeast preferential codons were used for Y₁ and Y₂, and other yeast elements were used to make the above formula as follows:

AOX1 Pm-yeast Ld (S)-Kex2 site-Y₁-AOX1Tm-AXO1 Pm-yeast Ld (S)-Kex2 site-Y₂-AOX1 Tm.

where the yeast Ld sequence translates into the signal sequence (pre) followed by the pro sequence. At the carboxy terminal of the pro sequence is the short sequence of “Lys-Arg” (“Kex2 site”) which is recognized by endoprotease Kex2 for cleavage.

1. Vector Construction

Briefly, the strategy for vector construction was:

Step 1: Based on yeast preferential codon, a fragment encoding human proinsulin analog B-C′-A flanked by recognition sites for restriction enzymes SnaBI and NotI, respectively, was cloned using PCR. “B-C′-A” is a proinsulin-like molecule where “C′” stands for an analog of the natural C-peptide found in proinsulin. Using the SnaBI and NotI restriction sites, the B-C′-A coding sequence was inserted into an expression cassette that was under the control of an AOX1 promoter in a pPIC9K plasmid. The resulting plasmid was termed “pPIC9K(B-C′-A).”

Step 2: Using pPIC9K(B-C′-A) as template and two primers with XhoI and EcoRI recognition sites on their respective ends, coding sequence for the B chain was cloned through PCR. The resulting fragment was then inserted into pPIC9K through the XhoI and EcoRI restriction sites, yielding a plasmid with an expression cassette that carries the coding sequence for the B chain. The plasmid was termed “pPIC9K(+B).”

Step 3: Step 2 was repeated by replacing B chain with A chain. The resulting plasmid was termed “pPIC9K(+A).”

Step 4: Using pPIC9K(+A) as template and two primers both with AatII recognition sites on their respective ends, the entire expression cassette including coding sequence for the A chain was cloned through PCR.

Step 5: The PCR product from Step 4 and pPIC9K(+B) from Step 2 were both treated with AatII and ligated, resulting in plasmid pPIC9K(+B+A) with two separate expression cassettes, one containing the coding sequence for the B chain and the other containing the coding sequence for the A chain.

Some of the steps may be performed in a different order, as would be apparent to one skilled in the art. Standard molecular biological procedures were followed in this example. See Ausubel, F. M., et al., Short Protocols in Molecular Biology, Second Edition (1992). Details of the procedure is as follows:

<Step 1: Construction of Intermediary Plasmid pPIC9K (B-C′-A)>

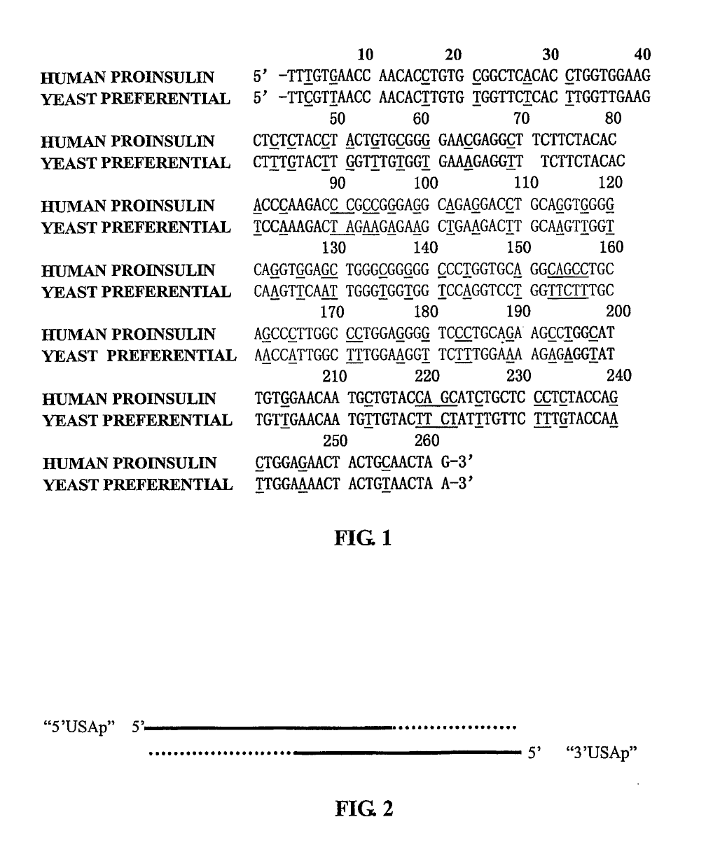

Referring to FIG. 1, natural DNA codons for human proinsulin (B-C-A) (from NCBI's online database), SEQ ID NO:1, and preferential codons by yeast, SEQ ID NO:2, are listed side-by-side with differences underlined. The Codons preferred by a particular prokaryotic or eukaryotic host (Murray, E. et al. Nuc. Acids Res. 17:477-508 (1989)) can be selected, e.g., using software programs such as DNAMAN, to increase the rate of heterologous polypeptide expression or to produce recombinant RNA transcripts having desirable properties, such as a longer half-life, than transcripts produced from naturally occurring sequence.

Still referring to FIG. 1, the 5′ portions of both sequences (in bold) encode the B chain, and the 3′ portions of both sequences (also in bold) encode the A chain. The middle portions encode the C-peptide.

To simplify the procedure, the C-peptide was shortened to C′, e.g., two amino acids encoded by “AAAAGA” as in this example. Of course, the C-peptide could be modified into a different length and/or of different amino acid content. Accordingly, two single-strand oligonucleotides, SEQ ID NOS:3 and 4, were designed to anneal to the coding sequence of B chain and A chain, respectively, and to include the C′ sequence. As a result, the pair of oligonucleotides can be used both as the template and as primers in a polymerase chain reaction (PCR) for the purpose of cloning the coding sequence for B-C′-A:

“5′-USAp” (100 nt) (SEQ ID NO:3):
5′-GCATTACGTATTCGTTAACCAACACTTGTGTGGTTCTCACTTGGTTG
AAGCTTTGTACTTGGTTTGTGGTGAAAGAGGTTTCTTCTACACTCCAAAG
ACT-3′,
and
“3′-USAp” (104 nt) (SEQ ID NO:4):
5′-ATATGCGGCCGCTTAGTTACAGTAGTTTTCCAATTGGTACAAAGAAC
AAATAGAAGTACAACATTGTTCAACAATACCTCTTTTAGTCTTTGGAGTG
TAGAAGA-3′

The last twenty nucleotides (underlined) at the 3′ end of each of the two oligonucleotides, 5′-USAp and 3′-USAp, are complementary to each other. As illustrated in FIG. 2, these complementary portions (in solid line) would anneal to each other and facilitate extension (in dotted line) in both directions in a PCR reaction. As a result, a double-strand DNA fragment coding for proinsulin analog B-C′-A was obtained. On the 5′ end of oligonucleotide 5′-USAp, there is a recognition site for restriction enzyme SnaBI which is double-underlined. On the 5′ end of oligonucleotide 3′-USAp, there is a recognition site for restriction enzyme NotI, also double-underlined.

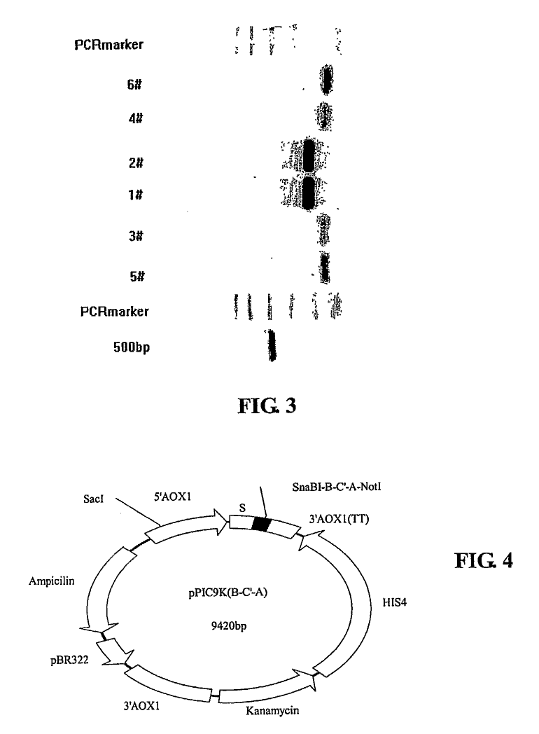

The two oligonucleotides, 5′-USAp and 3′-USAp, were manufactured by Integrated DNA Technologies Inc. (Coralville, Iowa, USA) through a custom order. To synthesize the B-C′-A fragment, the following were added in succession to a 0.5 ml PCR tube pre-chilled on ice: 5 μl of 10× buffer, 8.5 μl of dNTP mixture which contained 1.25 mM of each nucleotide, 10 μl of 5′USAp (50-100 ng), 10 μl of 3′USAp (50-100 ng), 0.5 μl of Taq DNA polymerase (5μ/μl), and ddH2O to 50 μl total volume. PCR reactions were then carried out in automated PCR equipment for 5 cycles of 4 min. at 94° C., 2 min. at 55° C. and 3 min. at 72° C. The size of any PCR product was examined by electrophoresis in 0.7% agarose gel. As shown in FIG. 3, an expected 184 bp fragment was obtained.

Yeast shuttle vector pPIC9K was purchased from Invitrogen, Calsbad, Calif., USA. Referring to FIG. 4, this vector carries a kanamycin resistance gene, an ampicillin resistance gene, and also a histidine marker. Therefore, cells, e.g., E. coli or yeast cells, transformed by this vector can be selected or screened on histidine-deficient medium for resistance to high level of kanamycin or/and ampicillin. The expression cassette in pPIC9K, which starts with a “5′AOX1” (promoter) and ends with “3′AOX1(TT)” (terminator), also includes a leader sequence “S” that directs the secretion of expressed proteins, and an endoprotease Kex2 recognition site towards the 3′ end of the leader sequence. “pBR322” 0 is the replication initiation site of E. coli plasmid pBR322.

Gel-purified B-C′-A coding sequence fragments and samples of plasmid pPIC9K were digested with both restriction enzymes SnaBI and NotI, and then ligated with DNA ligase. The product, an intermediary plasmid pPIC9K (B-C′-A) depicted in FIG. 4, was transformed into TOP10F′ (E. coli) cells. Then positive recombinants were selected and insertion of the correct B-C′-A sequence fragment was verified through restriction enzyme analysis and electrophoresis.

Selected positive recombinants were further sequenced by TaKaRa Biotechnology (Dalian) Co., Ltd. (Dalian, China) and the results showed the insertion as follows (SEQ ID NO:5):

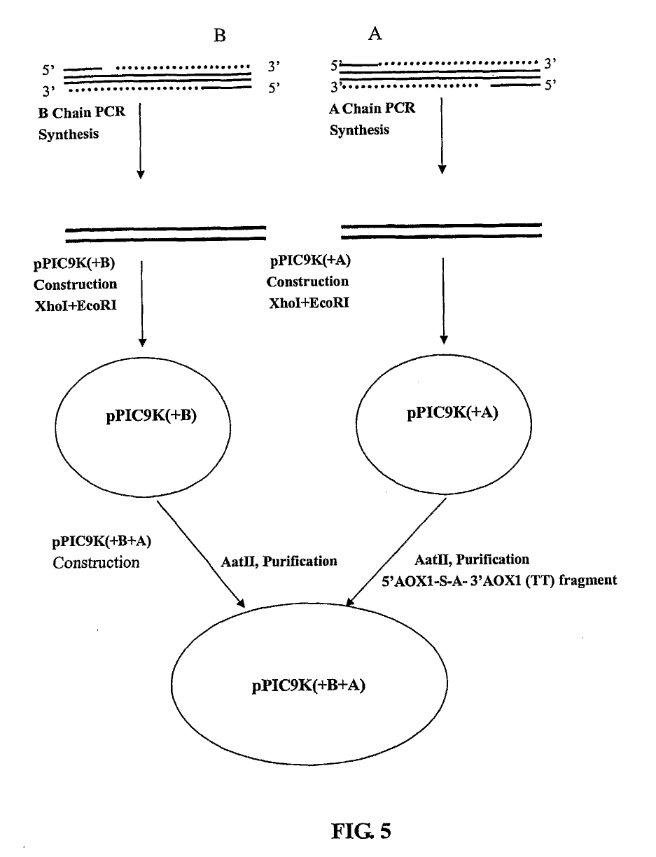

5′-GCATTACGTATTCGTTAACCAACACTTGTGTGGTTCTCACTTGGTTG
AAGCTTTGTACTTGGTTTGTGGTGAAAGAGGTTTCTTCTACACTCCAAAG
ACTAAAAGAGGTATTGTTGAACAATGTTGTACTTCTATTTGTTCTTTGTA
CCAATTGGAAAACTACTGTAACTAAGCGGCCGCATAT-3′

where the SnaBI recognition site on the 5′ end and the NotI site on the 3′ end are both underlined, and the C′-peptide coding sequence is double-underlined. A homologous sequence comparison and matching analysis using DNAMAN software verified that the PCR product's sequence is completely identical to the expected B-C′-A coding sequence. FIG. 5 illustrates how to eventually construct pPIC9K(+B+A) from pPIC9K(B-C′-A). The steps shown in FIG. 5 are explained in more detail below as Steps 2-5 in this example.
<Step 2: Construction of Intermediary Plasmid pPIC9K(+B)>

The following oligonucleotides were ordered through TaKaRa Biotechnology and used as primers in PCR against pPIC9K(B-C′-A) as the template:

5′-primer (36 nt) (SEQ ID NO:6):
5′-ATCTCTCGAGAAAAGATTCGTTAACCAAC ACTTGTG-3′
3′-primer (36 nt) (SEQ ID NO:7):
5′-ATCTGAATTCATCTTAAGTCTTTGGAGTGTAGAAGA-3′

where the XhoI recognition site on the 5′-primer and the EcoRI site on the 3′-primer are both underlined.

Under the above-described PCR conditions, a product of 122 bp including the code for B chain was obtained after 30 reaction cycles. The PCR product was of the following sequence (SEQ ID NO:8) where the XhoI and EcoRI restriction enzyme sites on the 5′ and 3′ ends, respectively, are underlined:

5′-ATCTCTCGAGAAAAGATTCGTTAACCAACACTTGTGTGGTTCTCACT
TGGTTGAAGCTTTGTACTTGGTTTGTGGTGAAAGAGGTTTCTTCTACACT
CCAAAGACTTAAGATGAATTCAGAT-3′

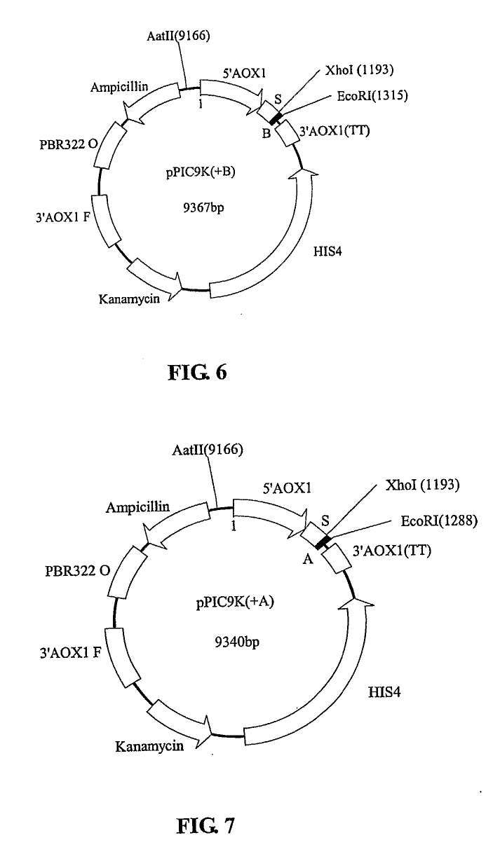

This PCR product was digested by both XhoI and EcoRI and purified. Then it was inserted into plasmid pPIC9K that had also been digested with XhoI and EcoRI, and ligated to form another intermediary vector pPIC9K (+B) as shown in FIG. 6. Positive recombinants were selected and verified through restriction enzyme analysis.

<Step 3: Construction of Intermediary Plasmid pPIC9K(+A)>

The following oligonucleotides were ordered through TaKaRa Biotechnology and used as primers in PCR against pPIC9K(B-C′-A) as the template:

5′-primer (36 nt) (SEQ ID NO:9):
5′-ATCTCTCGAGAAAAGAGGTATTGTTGAACAATGTTG-3′
3′-primer (36 nt) (SEQ ID NO:10):
5′-ATCTGAATTCATCTAGTTACAGTTAGTTTTCCAATT-3′

where the XhoI recognition site on the 5′-primer and the EcoRI site on the 3′-primer are both underlined.

Under the above-described PCR conditions, a product of 95 bp including the code for A chain was obtained after 30 reaction cycles. The PCR product was of the following sequence (SEQ ID NO:11) where the XhoI and EcoRI restriction sites on the 5′ and 3′ ends, respectively, are underlined:

5′-ATCTCTCGAGAAAAGAGGTATTGTTGAACAATGTTGTACTTCTATTT
GTTCTTTGTACCAATTGGAAAACTACTGTAACTAGATGAATTCAGAT-3′

This PCR product was digested by both XhoI and EcoRI and purified. Then it was inserted into plasmid pPIC9K that had also been digested with XhoI and EcoRI and ligated into another intermediary vector pPIC9K (+A) shown in FIG. 7. Positive recombinants were selected and verified through restriction enzyme analysis.

<Step 4: Cloning of an Expression Cassette that Expresses the Coding Sequence for the A Chain>

The following oligonucleotides were designed to anneal to the two ends of the expression cassette in pPIC9K(+A). The oligonucleotides were ordered through TaKaRa Biotechnology and used as primers in PCR against pPIC9K(+A) as the template:

5′-primer (30 nt) (SEQ ID NO:12):
5′-ATCTGACGTCAGATCTAACATCCAAAGACC-3′
3′-primer (30 nt) (SEQ ID NO:13):
5′-ATCTGACGTCAAGCTTGCACAAACGAACTT-3′

where the Aat II recognition sites on both the 5′-primer and the 3′-primer are underlined.

Following the above-described PCR conditions for 30 reaction cycles, a PCR product was verified through agarose gel electrophoresis. The gel-purified PCR product was further treated with AatII restriction enzyme to yield a 1697 bp fragment of the following structure:

AatII-5′AOX1 promoter-S-Insert A-3′AOX1(TT)-AatII

<Step 5: Construction of Final Plasmid pPIC9K(+B+A)>

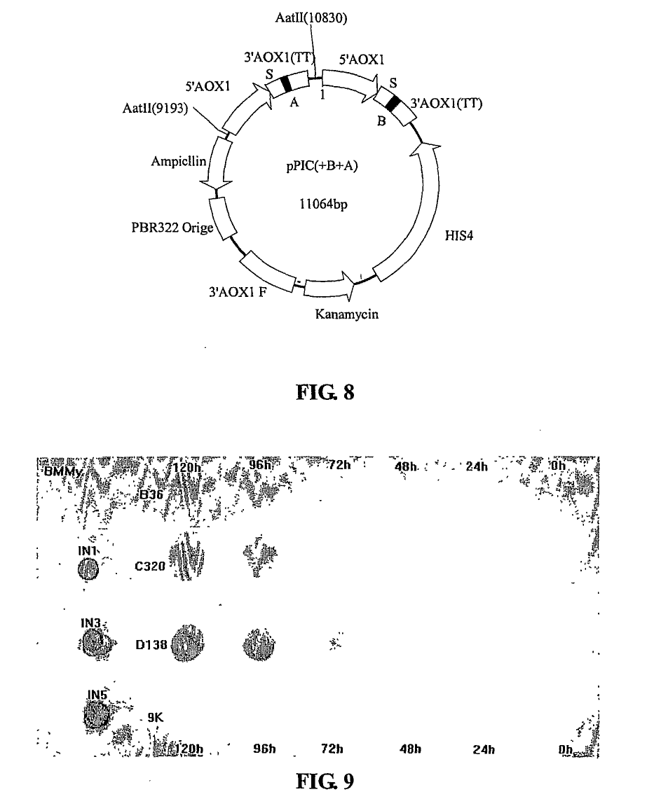

Referring now to FIG. 8, The 1697 bp 5′-AOX1-S-InsA-3′AOX1 (TT) fragment obtained from the previous step was ligated with AatII-treated and purified pPIC9K(+B) (from Step 2). After transformation and selection, the final expression vector pPIC9K(+B+A) was obtained. Positive recombinants were selected and verified through restriction enzyme analysis.

2. Host Cell Cultivation, Transformation and Characterization of Recombinants

All methods in this example regarding host cell cultivation, transformation and characterization of the recombinants followed the recommendation in Pichia Protocols, edited by D. Higgins and J. Cregg, Humana Press (1998). Briefly, the procedures included culturing P. pastoris cells GS115, transformation, recombinant selection, phenotype (−his +glucose/−his and +methanol) validation, the selection and expression of Mutu phenotype, and selection of high expression clones in shake-flask or a fermentation tank.

Specifically, the protocol for transformation (electroporation) and selection for the positive recombinants was as follows: competent GS115.2 cells were prepared. DNA vector pPIC9K(+B+A) linearized by SalI was added to the cell culture. After having been mixed well, the cell culture was transferred to a 0.2 cuvette. Electroporation was carried out under conditions as follows: 1.5 KV, 2.5 μF, and 200 ohm. After electroporation, T1 and T2 should display between 4 to 5. Cells were then washed with 1.0 ml of 1.0 M sorbitol, and selected aliquots were spread on agar plates containing MD/-HIS, and incubated for 24 hours at 30° C. before visual inspection for yeast colonies. Single colonies from the plates were picked and spread onto selection plates with glucose (MD) or methanol (MM), respectively. The fast growing colonies on MM plates were Mut+, others were Muts which grow slowly on methanol. Single colonies were spread onto plates for a second time with different concentrations of antibiotic G418, and colonies that exhibited the greatest level of resistance to G418, i.e., with the highest number of copies of transformed vectors, were kept for use.

P. pastoris cell lines (GSGT1) successfully transformed with expression vector pPIC9K(+B+A) were registered and deposited in China Center for Type Culture Collection (CCTCC) at Wuhan University, Wuhan, China.

3. Expression of Human Insulin

P. pastoris cells from a single colony containing high copies of recombinant vectors were incubated in 25 ml MGY medium overnight at 30° C. while being shaken at 300 rpm (OD₆₀₀=about 4). These cells were then transferred into 1 L MGY medium at 30° C. and at 300 rpm (OD₆₀₀=about 4) until the cell growth curve entered the log phase. The cells were harvested through centrifugation at 2500×g, room temperature for 5 minutes. The resulting cell pellet was re-suspended in a proper volume of BMMY medium (OD₆₀₀=1.0) and continued to grow at 30° C. and at 300 rpm for another 90 hours. Daily addition of 100% methanol was provided to the cell culture to keep methanol concentration at 0.5% (V/V) for inducing expression of the A and B chains of human insulin. The methanol concentration in the shaker was determined by gas-phase chromatography (GPC) every 4 hours until 90 hours. The resulting cell culture medium was then subjected to centrifugation at 2500×g under room temperature for 5 minutes to pellet the cells. The supernatant was collected and stored at −80° C. for later use. Host cells may be repeatedly cultured in fresh media to form additional batches of supernatant from each culture, and insulin may be isolated from each batch of supernatant.

Expressed proteins in the supernatant were concentrated after precipitation through at least one of micro filtration, hydrophobic interaction chromatography, and ion exchange chromatography. Purification of the expressed insulin from the above supernatant was achieved through reverse-phase HPLC.

4. Product Characterization

The above supernatant products were concentrated by protein precipitation, and expression of human insulin was confirmed by Immunodotting Blot and Western Blot. Referring to FIG. 9, the results of an Immunodotting Blot are presented in a diagram. Following standard Immunodotting Blot protocol, monoclonal mouse anti-insulin antibody (Code No.: 2D11-H5, Santa Cruz Biotechnology, Inc., Santa Cruz, Calif., USA) was used. The far left column were positive controls of increasing concentrations of standard human insulin from IN1 to IN5. Each of the remaining columns represents the cell medium collected at various hours from selected yeast colonies transformed by the recombinant vector pPIC9K(+B+A). Of yeast colonies listed in different rows, “9K” was the negative control with medium collected from cells transformed with the original plasmid pPIC9K. As FIG. 9 shows, all three positive colonies B36, C320, and D138 exhibited increasing presence of human insulin as fermentation time increased.

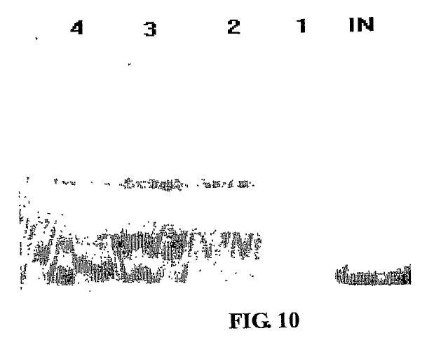

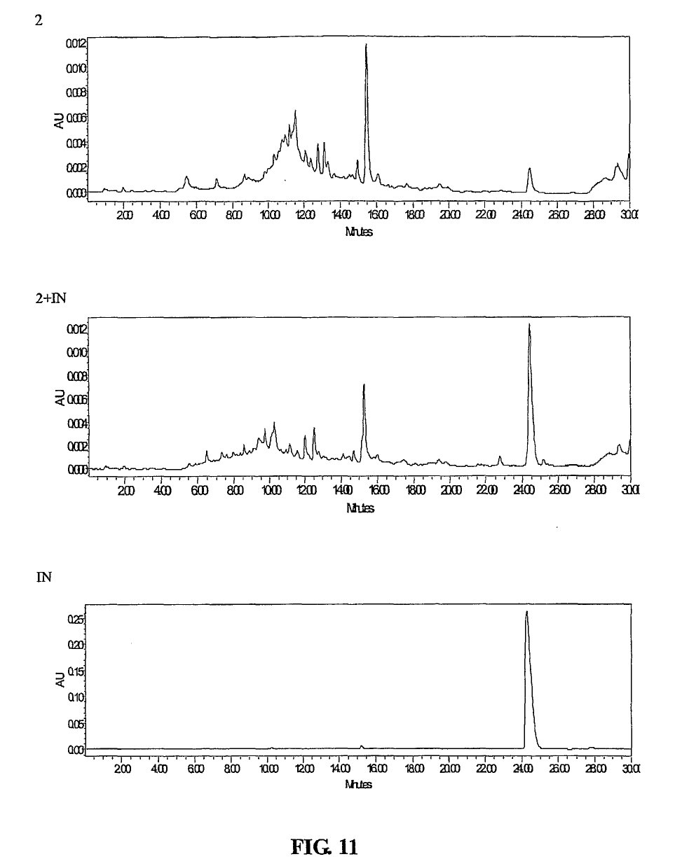

Referring to FIG. 10, results of a Western Blot are presented through a photographic image. Following standard Western Blot protocol, monoclonal mouse anti-insulin antibody (Code No.: 2D11-H5) was used. The far right lane labeled as “IN” contained a human insulin standard. And the rest of the lanes represented samples from culture media of selected yeast colonies transformed by the recombinant vector pPIC9K(+B+A). Specifically, lanes 1-4 were samples collected at 24, 48, 72, and 96 hours of fermentation, respectively. A visible protein band matching that of the human insulin standard started to emerge at hour 72 in the experiment pictured in FIG. 10, confirming the expression of a protein of the expected molecular weight. Together with FIG. 9, expression, assembly, and secretion of human insulin's B and A chains by the transformed Pichia pastoris cells were confirmed.

Referring to FIG. 11, the elution profile and expression levels of insulin were determined by High Performance Liquid Chromatography (HPLC) analysis. All three figures had a common peak at 24.5 minutes. Therefore, the retention took 24.5 minutes, which was exactly the same with the internal insulin standard. The yield in the tested system was about 400 mg/L compared with standard. The fractions were further analyzed for insulin by SDS-PAGE analysis (not shown) and by Western Blot analysis (FIG. 10).

There are numerous ways, which ought to be apparent to one skilled in the art, that can assay the bioactivity and function of the expressed insulin. For example, in one assay, 0.2 ml of purified expression product is injected into each of 40 healthy and normal mice by ip (intra peritoneal). After 20 minutes, blood samples are taken from these mice as a test group. After 3 hours, when the blood sugar levels in these mice return to normal, 0.2 ml commercial human insulin is injected into each of the same 40 mice by ip (intra peritoneal injection). After another 20 minutes, blood samples are collected from these mice as positive control group. Blood samples of these mice collected under normal condition before any of the aforementioned injections (of either the expression product or commercial insulin) constitute the negative control group. The blood sugar level of all three sample groups are examined and biological statistical analysis is performed. If the test group shows reduction in the blood sugar level, the bioactivity is proved and the i.u. (international units) of the administered test product can be calculated by comparing against the known i.u. of the positive group.

### EXAMPLE 2

This example describes an alternative method for constructing the recombinant expression vector pPIC9K(+B+A). Specifically, the step of constructing the intermediary vector pPIC9K(B-C′-A) in Example 1 is omitted. This example is described to highlight its differences from Example 1. Recitations of similarities are hereby omitted.

<Step 1: Construction of Intermediary Plasmid pPIC9K (+B)>

Based on yeast preferential codon for human proinsulin depicted in FIG. 1, a DNA fragment encoding the B chain of human insulin is obtained as follows: Two oligonucleotides are each designed to anneal to an end of B chain's coding sequence:

5′-Oligo (71 nt) (SEQ ID NO:14):
5′-GCTACTCGAGAAAAGATTCGTTAACCAACACTTGTGTGGTTCTCACT
TGGTTGAAGCTTTGTACTTGGTTT-3′
3′-Oligo (70 nt) (SEQ ID NO:15):
5′-TAGCGCGGCCGCTTAAGTCTTTGGAGTGTAGAAGAAACCTCTTTCAC
CACAAACCAAGTACAAAGCTTCA-3′

The last twenty nucleotides (underlined) at the 3′ end of each of the two oligonucleotides, SEQ ID NOS:14 and 15, are complementary to each other. Similar to the oligonucleotides depicted in FIG. 2, the complementary portions (in solid line) anneal to each other and facilitate extension (in dotted line) in both directions in a PCR reaction. As a result, a double-strand DNA fragment encoding the B chain is obtained. On the 5′ end of SEQ ID NO:14, there is a recognition sequence for restriction enzyme XhoI which is double-underlined. On the 5′ end of SEQ ID NO:15, there is a recognition sequence for enzyme NotI, also double-underlined.

PCR reactions are carried out under conditions specified in Example 1 for 5 cycles to yield an expected 121 bp fragment. This fragment is then inserted into the carrying plasmid pPIC9K to construct an intermediary vector pPIC9K(+B). Specifically, gel-purified B chain sequence fragments obtained from PCR are digested with both XhoI and NotI. Plasmid pPIC9K is completely digested with NotI first and then partially digested with XhoI. The larger fragment of the digested plasmid is gel-purified from the digestion mixture and ligated with the digested B chain sequence fragment using DNA ligase. The ligated product is transformed into TOP10F′ (E. coli) cells and copies of intermediary plasmid pPIC9K(+B) are made by the E. coli cells.

Positive recombinant is selected and successful insertion is verified through restriction enzyme analysis and electrophoresis. Further, the digested fragment, i.e., the inserted coding sequence for the B chain, is sequenced to check against the expected sequence as follows (SEQ ID NO:16):

5′-TTCGTTAACCAACACTTGTGTGGTTCTCACTTGGTTGAAGCTTTGTA
CTTGGTTTGTGGTGAAAGAGGTTTCTTCTACACTCCAAAGACT-3′

<Step 2: Construction of Intermediary plasmid pPIC9K (+A)>

Similarly to Step 1 of this example, the following pair of oligonucleotides are designed and custom-made based on yeast preferential codons for the A chain of human proinsulin depicted in FIG. 1:

5′-Oligo (90 nt) (SEQ ID NO:17):
5′-GCTACTCGAGAAAAGAGGTATTGTTGAACAATGTTGTACTTCTATTT
GTTCTTTGTACCAATTGGAAAACTACTGTAAGCGGCCGCGCTA-3′
3′-Oligo (90 nt) (SEQ ID NO:18):
5′-TAGCGCGGCCGCTTACAGTAGTTTTCCAATTGGTACAAAGAACAAAT
AGAAGTACAACATTGTTCAACAATACCTCTTTTCTCGAGTAGC-3′

Recognition sequences for restriction enzyme XhoI on the 5′ end of SEQ ID NO:17 and for NotI on the 5′ end of SEQ ID NO:18, respectively, are double-underlined.

PCR reactions are carried out as outlined in Step 1 of this example to yield an expected 91 bp fragment. The fragment is cut and pasted into the pPIC9K using restriction enzymes XhoI and NotI, and DNA ligase. After transforming TOP10F′ cells with the expected pPIC9K (+A) plasmid, identity of the inserted sequence is verified as described above.

Step 3: Cloning of an expression cassette that expresses the coding sequence for the A chain is the same as Step 4 of Example 1.

Step 4: Construction of the final plasmid pPIC9K (+B+A) is the same as Step 5 of Example 1.

### EXAMPLE 3

Similar to protocols illustrated by Examples 1 and 2, an expression vector, e.g., a yeast vector, can be constructed to include two separate expression cassettes for the purpose of expressing and assembling in vivo two subunits of a heterodimer, in this case, IL-12. Specifically, one expression cassette is constructed to include the coding sequence for p35 of IL-12, and the other to include the coding sequence for p40 also of IL-12.

The same carrier vector and host organism can be used for the purpose of making IL-12. Basically, one may modify Example 1 and 2 to produce IL-12 by simply substituting the coding sequence for p35 for its counterpart for the B chain of human insulin, and then the coding sequence for p40 for its counterpart for the A chain of human insulin. Details of such modification are well within the knowledge of one skilled in the art.

Each of the patent documents and scientific publications disclosed hereinabove is incorporated by reference herein for all purposes.

While the invention has been described with certain embodiments so that aspects thereof may be more fully understood and appreciated, it is not intended to limit the invention to these particular embodiments. On the contrary, it is intended to cover all alternatives, modifications and equivalents as may be included within the scope of the invention as defined by the appended claims.

What is claimed is:

1-31. (canceled)

32. A recombinant DNA comprising the sequence of:
Pm₁-Ld₁-Pt₁-Y₁-Tm₁-Pm₂-Ld₂-Pt₂-Y₂-Tm₂,
where each listed element is operably linked to an adjacent element, Pm stands for a yeast promoter sequence, Ld stands for a yeast leader sequence, Pt stands for a protease recognition sequence, and Tm stands for a yeast termination sequence.

33. The recombinant DNA of claim 32 wherein the yeast is Pichia Pastoris.

34. The recombinant DNA of claim 32 wherein at least one of the Pt₁ and Pt₂ comprises a codon for amino acid lysine followed by a codon for amino acid Arginine.

35. The recombinant DNA of claim 32 wherein Y₁ stands for sequence for one of the B and A chains of human insulin while Y₂ stands for sequence for the other one of the B and A chains of human insulin.

36. The recombinant DNA of claim 32 wherein Y₁ and Y₂ stand for DNA sequences for two subunits of a cytokine, respectively.

37. The recombinant DNA of claim 36 wherein the cytokine is interleukin-12.

38. A recombinant human insulin molecule produced by:
(a) providing a eukaryotic cell comprising a recombinant genetic construct that comprises a first expression cassette and a second expression cassette, the first expression cassette comprising a sequence substantially corresponding to the A chain of the human insulin molecule, the second expression cassette comprising a sequence substantially corresponding to the B chain of the human insulin molecule;
(b) inducing the cell to express, through the recombinant genetic construct in the cell, the recombinant human insulin molecule and to secrete the expressed recombinant human insulin molecule into a surrounding culture; and
(c) harvesting the secreted human insulin molecule from the surrounding culture.

39. The recombinant human insulin molecule of claim 38 wherein the recombinant human insulin molecule secreted in step (b) is bioactive.

40. The recombinant human insulin molecule of claim 38 wherein the eukaryotic cell is a yeast cell.

41. The recombinant human insulin of claim 40 wherein the yeast cell is of a strain selected from the group consisting of Pichia, Hansenula, Candida, and Torulopsis.

42. The recombinant human insulin of claim 41 wherein the yeast cell is Pichia pastoris.

43. The recombinant human insulin of claim 40 wherein the yeast cell is methylotrophic.

44. The recombinant human insulin of claim 38 wherein the recombinant genetic construct comprises the sequence of:
Pm₁-Ld₁-Pt₁-Y₁-Tm₁-Pm₂-Ld₂-Pt₂-Y₂-Tm₂,
where each listed element is operably linked to an adjacent element, Pm stands for a promoter sequence, Ld stands for a leader sequence, Pt stands for a protease recognition sequence, Tm stands for a termination sequence, and where Y₁ stands for sequence for one of the B and A chains of human insulin while Y₂ stands for sequence for the other one of the B and A chains of human insulin.

45. The recombinant human insulin of claim 44 wherein Pm₁ and Pm₂ are substantially the same, Ld₁ and Ld₂ are substantially the same, Pt₁ and Pt₂ are substantially the same, and Tm₁ and Tm₂ are substantially the same.

46. The recombinant human insulin of claim 44 wherein in the sequence for the recombinant genetic construct, Pm stands for a yeast promoter sequence, Ld stands for a yeast leader sequence, and Tm stands for a yeast termination sequence.

47. The recombinant human insulin of claim 44 wherein the protease recognition sequence encodes for a Kex2 processing site.

48. The recombinant human insulin of claim 44 wherein the leader sequence encodes a signal that leads a polypeptide that is expressed by the recombinant genetic construct through a secretory pathway of the cell.

49. A method for producing human recombinant insulin comprising the steps of:
(a) providing a eukaryotic cell comprising a recombinant genetic construct that comprises a first expression cassette and a second expression cassette, the first expression cassette comprising a sequence substantially corresponding to the A chain of the human insulin molecule, the second expression cassette comprising a sequence substantially corresponding to the B chain of the human insulin molecule;
(b) inducing the cell to express, through the recombinant genetic construct in the cell, the recombinant human insulin molecule and to secrete the expressed recombinant human insulin molecule into a surrounding culture; and
(c) harvesting the secreted human insulin molecule from the surrounding culture.

50. The method of claim 49 wherein step (b) comprises inducing the cell to secrete the recombinant human insulin molecule secreted in its bioactive form.

51. The method of claim 49 wherein the eukaryotic cell is a yeast cell.

52. The method of claim 51 wherein the yeast cell is of a strain selected from the group consisting of Pichia, Hansenula, Candida, and Torulopsis.

53. The method of claim 51 wherein the yeast cell is Pichia pastoris.

54. The method of claim 51 wherein the yeast cell is methylotrophic.
